(19) United States
(12) Patent Application Publication
(10) Pub. No.: US 2010/0260813 A1
(43) Pub. Date: Oct. 14, 2010

(54) Rodenticide Mixture

(75) Inventors: Gerhard Schnabel, Elsenfeld (DE); Michael Ishaque, Mannheim (DE); Dieter Zeller, Speyer (DE); Lars Weichel, Maikammer (DE); Andrej Brejc, Weisenheim am Berg (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/680,651

(22) PCT Filed: Sep. 30, 2008
(86) PCT No.: PCT/EP08/63101

§ 371 Date: Mar. 29, 2010

(30) Foreign Application Priority Data
Oct. 1, 2007 (EP) 07117613.5

(51) Int. Cl. A01N 25/34; A01N 25/00; A01N 25/08; A01P 11/00; A01N 43/16; A01N 43/18; A01N 59/22; A01N 45/00; A01N 37/18; A01N 43/42; A01N 33/02; A61K 31/60; A01N 43/00; A61K 31/192; A01N 37/10; A01N 43/72; A01N 31/08; A01N 43/40; A01N 43/56; A01N 43/62; A01N 43/50; A61K 31/075; A01N 31/00; A01N 43/84; A01N 59/02

(52) U.S. Cl. 424/408; 424/84; 424/410; 424/409; 514/457; 514/432; 424/623; 514/167; 514/628; 514/282; 514/646; 514/279; 514/159; 514/161; 514/570; 514/569; 514/226.5; 514/731; 514/329; 514/406; 514/221; 514/220; 514/401; 514/721; 514/53; 514/724; 514/730; 514/231.2; 424/712; 514/22

(57) ABSTRACT

A rodenticidal mixture comprising
a) 0.0015 to 99.89% by weight of at least one rodenticide,
b) 10⁻⁸ to 95% by weight of at least one painkiller and
c) 0.01 to 99.9984% by weight of at least one biocide,
   where the percentages by weight are based on the total of components (a), (b) and (c), is suitable for the preparation of rodenticidal bait formulations which are employed for controlling rodent pests.

The invention relates to the field of pesticides, in particular to a rodenticidal mixture, a rodenticidal bait formulation comprising such a mixture, processes of preparing them, methods of using them, and a method of controlling rodent pests.

Rodent pests such as mice and rats cause considerable economical damage by devastating and soiling stored products and destroying buildings, electrical goods and useful areas. Moreover, mice and rats transmit a large number of infectious diseases such as plague, typhoid, leptospirosis, trichinosis and salmonellosis. The effective control of these rodent pests is therefore indispensable.

Ethical considerations in this context require that no unnecessary suffering and harm be caused to the animals which are being controlled. In the European Union, this sentiment is reflected for example in Directive 91/414 EEC for the licensing of new pesticides.

Anticoagulants such as coumarin and indandione derivatives play an important role in the control of rodents. These active ingredients are simple to handle, relatively harmless to humans and have the advantage that, as the result of the delayed onset of the activity, the animals being controlled identify no connection with the bait which they have ingested, avoiding it. This is an important aspect in particular in social animals such as rats, where individuals act as tasters.

Since in many cases the animals die as the result of internal bleeding, however, it is held in the literature that it is precisely this important class of rodenticides which cannot be considered to belong to the category of the humane preparations, i.e. those which act with as little pain as possible. (PSD (Pesticide Safety Directorate) 1997 Assessment of Humaneness of Vertebrate Control Agents—Evaluation of Fully Approved or Provisionally Approved Products, No. 171 (December 1997). Pesticides Safety Directorate: York, UK).

The abovementioned document proposes to examine the possibility of using anesthetics and analgesics in rodenticidal formulations comprising anticoagulants.

Some isolated studies in this respect have been conducted. Thus, K. E. Littin and C. E. O'Connor report on the simultaneous administration of rodenticides and analgesics (in: Ramus A, Pinheiro Machado Filho L C and Hötzel M J (eds) Proceedings of the 34th International Congress of The International Society of Applied Ethology p. 159. Federal University of Santa Catarina: Florianopolis, Brazil).

C. A. Macks et al. studied the combination of the rodenticide 1080 (sodium fluoroacetate) with carprofen, diazepan and clonidine (Wildlife Research 27 (2000), 483-494). Moreover, it has been proposed on use anticoagulants in connection with substances which reduce pain and simultaneously enhance the activity of the rodenticide, for example salicylates (Timm R M 1994a Description of active ingredients. Cooperative Extension Division, Institute of Agriculture and Natural Resources, University of Nebraska: Lincol, USA).

However, these studies have not found a practical application to date. This is not surprising since the demands made on commercially utilizable rodenticidal bait formulations are very stringent, for example as regards activity, environmental stability, the possibility of simple preparation, storage and handling, low toxicity to humans and economical aspects.

It was therefore an object of the invention to provide rodenticidal bait formulations which are suitable for practice conditions and which make possible the control of rodent pests which is less painful for the animals concerned.

The object is achieved by a rodenticidal mixture which, besides a rodenticide, comprises a painkiller and a biocide in a specific ratio.

The present invention therefore relates to a rodenticidal mixture comprising
a) 0.0015 to 99.89% by weight of at least one rodenticide,
b) 10⁻⁸ to 95% by weight of at least one painkiller and
c) 0.1 to 99.9984% by weight of at least one biocide,
   where the percentages by weight are based on the total of components (a), (b) and (c).

The mixture according to the invention shows excellent stability and is outstandingly suitable for the effective control of rodent pests.

For the purposes of the invention, the term rodenticide means a solid or liquid active ingredient which is suitable for controlling rodent pests, preferably rodents, in particular rats and mice.

Suitable classes of rodenticides comprise:
(aa) anticoagulants, in particular coumarin derivatives such as brodifacoum (a1), bromadiolone (a2), coumachlor (a3), coumafuryl (a4), coumatetralyl (a5), difenacoum (a6), difethialone (a7), flocoumafen (a8) and warfarin (a9), indandione derivatives such as chlorophacinone (a10), diphacinone (a11) and pindone (a12),
(ab) inorganic rodenticides such as arsenic oxide (a13), phosphorus (a14), potassium arsenite (a15), sodium arsenite (a16), thallium sulfate (a17) and zinc phosphide (a18);
(ac) organochlorine rodenticides such as γ-HCH (a19), HCH (a20) and lindan (a21);
(ad) organophosphorus rodenticides such as phosacetim (a22);
(ae) pyrimidineamine rodenticides such as crimidine (a23);
(af) thiourea rodenticides such as antu (a24);
(ag) urea rodenticides such as pyrinuron (a25);
(ah) garden rodenticides such as scilliroside (a26) and strychnine (a27);
(ai) unclassified rodenticides such as bromethalin (a28), chloralose (a29), α-chlorohydrin (a30), ergocalciferol (a31), fluroroacetamide (a32), flupropadine (a33), norbormide (a34), sodium fluoroacetate (a35) and vitamin D3 (a36).

Preferred rodenticides are anticoagulants, in particular coumarin derivatives, particularly preferably flocoumafen and difenacoum. Furthermore preferred are mixtures of an anticoagulant with ergocalciferol or vitamin D3.

Zinc phosphide is equally preferred.

The rodenticide component (a) in the mixture according to the invention amounts to preferably 0.015 to 99.5% by weight, especially preferably 0.15 to 99% by weight.

For the purposes of the invention, the term painkiller refers to an active ingredient which reduces the toxicity symptoms caused by the rodenticide in the rodent pest concerned, thereby comprising traditional painkillers (analgesics), but also sedatives, anxiolytics and antidepressants.

Suitable analgesics are opioid analgesics and non-opioid analgesics. Examples of suitable opioid analgesics are morphine (b1), codeine (b2), dihydrocodeine (b3), hydromorphone (b4), oxycodone (b5), hydrocodone (b6), pethidine (b7), levomethadone (b8), levacetylmethadol (b9), piritramide (b10), pentazocine (b11), buprenorphine (b12), nalbuphine (b13), tilidine (b14), tramadol (b15) and methadone (b16).

Non-opioid analgesics comprise non-steroidal anti-inflammatory drugs (NSAIDs), non-acidic antipyretic analgesics, pyrazole derivatives and compounds which do not inhibit prostaglandin synthesis. Suitable non-steroidal anti-inflammatories are, in particular, salicylic acid derivatives such as acetylsalicylic acid (b17), amides of salicylic acid (b18), salsalate (b19) and diflunisal (b20), acetic acid derivatives such as indometacin (b21), acemetacin (b22), diclofenac (b23) and lonazolac (b24), propionic acid derivatives such as ibuprofen (b25), flurbiprofen (b26), ketoprofen (b27), dexketoprofen (b28), naproxen (b29) and tiaprofen acid (b30), oxicams such as piroxicam (b31), tenoxicam (b32), meloxicam (b33) and lornoxicam (b34), anthranilic acid derivatives such as mefenamic acid (b35) and flufenamic acid (b36), aniline derivatives such as paracetamol (b37), pyrazolinone derivatives such as phenazone (b38), propyphenazone (b39) and metamizole (b40) and also nabumetone (b41), azapropazone (b42), aceclofenac (b43) and oxaceprol (b44) and COX-2-selective non-steroidal anti-inflammatories such as rofecoxib (b45) and celecoxib (b46).

Non-opioid analgesics which do not inhibit prostaglandin synthesis comprise nefopam (b47) and flupirtin (b48).

Examples of suitable sedatives are narcotics such as propofol (b49), neuroleptics (b50) such as promethazine, opioids such as fentanyl (b51) and sulfentanil (b52), α-2-adrenoreceptor agonists such as clonidine (b53), barbiturates such as phenobarbital (b54) and pentobarbital (b55) and benzodiazepines which are mentioned among the anxiolytics.

Suitable anxiolytics comprise benzodiazepines such as alprazolam (b56), bromazepam (b57), brotizolam (b58), chlordiazepoxide (b59), clobazam (b60), clonazepam (b61), diazepam (b62), clorazepate (b63), flunitrazepam (b64), flurazepam (b65), loprazolam (b66), lorazepam (b67), lormetazepam (b68), medazepam (b69), midazolam (b70), nitrazepam (b71), nordazepam (b72), oxazepam (b73), prazepam (b74), temazepam (b75), tetrazepam (b76) and triazolam (b77).

Suitable antidepressants are, for example, tricyclic antidepressants such as doxepin (b78), imipramin (b79), clomipramin (b80), amitriptylin (b81), trimipramin (b82) and opipramol (b83), selective serotonin reuptake inhibitors such as fluvoxamin (b84), fluoxetin (b85), citalopram (b86), escitalopram (b87), sertralin (b88) and paroxetin (b89), selective serotonin/noradrenaline reuptake inhibitors such as venlafaxin (b90), duloxetin (b91) and milnacipran (b92), selective noradrenaline reuptake inhibitors such as reboxetin (b93) and viloxazin (b94), MAO inhibitors such as moclobemid (b95) and tranylcypromin (b96), noradrenergic and specifically serotonergic antidepressants such as mirtazapin (b97), dual serotonergic antidepressants such as nefazodon (b98), dopamine reuptake inhibitors such as amineptin (b99) and methylphenidate (b100), serotonin reuptake enhancers such as thianeptine (b101) and phytopharmaceuticals such as kavapyrone (b102).

The abovementioned painkillers are commercially available and described for example in Mutschler, Arzneimittelwirkungen [Drug actions], 8th edition, Wissenschaftliche Verlagsgesellschaft mbH, Stuttgart 2001.

Preferred painkillers are analgesics and sedatives. Furthermore preferred are mixtures of analgesics and sedatives.

Preferred analgesics are morphine, codeine, dihydrocodeine, hydromorphine, oxycodone, pethidine, tramadol, methadone, acetylsalicylic acid, diflunisal, naproxen, proxicam, tenoxicam, meloxicam, paracetamol and phenazone.

Preferred sedatives are propofol, clonidine, barbiturates such as phenobarbital and pentobarbital and benzodiazepines, in particular those which have been mentioned among the anxiolytics.

Preferred mixtures of analgesics and sedatives are mixtures comprising one or more analgesics from the group consisting of morphine, codeine, dihydrocodeine, hydromorphine, oxycodone, pethidine, tramadol, methadone, acetylsalicylic acid, diflunisal, naproxen, proxicam, tenoxicam, meloxicam, paracetamol and phenazone and one or more sedatives from the group consisting of propotol, clonidine, phenobarbital, pentobarbital, alprazolam, bromazepam, brotizolam, chlordiazepoxide, clobazam, clonazepam, diazepam, clorazepate, flunitrazepam, flurazepam, loprazolam, lorazepam, lormetazepam, medazepam, midazolam, nitrazepam, nordazepam, oxazepam, prazepam, temazepam, tetrazepam and triazolam.

It is also possible for one and the same compound to act as rodenticide (a) and painkiller (b). Examples of such compounds are barbiturates which, when present in a higher dosage rate, may not only have the desired sedative effect, but may also be lethal.

The painkiller preferably amounts to 10⁻⁷ to 75% by weight, especially preferably 10⁻⁶ to 50% by weight, in particular 10⁻⁵ to 50% by weight, in the mixture according to the invention.

For the purposes of the invention, the term biocide refers to a disinfectant or preservative which is active against viruses, microbes, fungi, harmful arthropods and/or helminths.

Examples of suitable biocides (c) are benzyl alcohol; 2,4-dichlorobenzyl alcohol; 2-phenoxyethanol; 2-phenoxyethanol hemiformal; phenyl ethyl alcohol; 5-bromo-5-nitro-1,3-dioxane; bronopol; formaldehyde; dimethyloldimethylhydantoin; glyoxal; glutaraldehyde; sorbic acid; benzoic acid; salicylic acid; p-hydroxybenzoic ester; chloroacetamide; N-methylolchloroacetamide; phenols such as p-chloro-m-cresol, o-phenylphenol and sodium o-phenylphenolate; 4,4-dimethyl-1,3-oxazolidine; 1,3,5-hexahydrotriazine derivatives; quaternary ammonium compounds such as N(C₁₂-C₁₈)-, (C₁₂-C₁₆)-, (C₁₂-C₁₄)-alkyl-N,N-dimethylbenzylammonium chloride, N-di-C₈-C₁₀-alkyldimethylammonium chloride, di-n-decyldimethylammonium chloride and C₁₂-C₁₄-alkyl[(ethylphenyl)methyl]dimethylammonium chloride; [2-[[2-[(carboxyethyl)(2-hydroxyethyl)amino]ethyl]amino]-2-oxoethyl]cocoalkyldimethyl hydroxides (internal salts); cetylpyridinium chloride; diguanidine; polybiguanide; chlorhexidine; 1,2-dibromo-2,4-dicyanobutane; 3,5-dichloro-4-hydroxybenzaldehyde; ethylene glycol hemiformal; tetra(hydroxymethyl)phosphonium salts; chlorophene; dichlorophene; 2,2-dibromo-3-nitrilopropionamide; 3-iodo-2-propynyl N-butylcarbamate; methyl N-benzimidazol-2-ylcarbamate; di-N-methyl-2,2′-dithiodibenzamide; 2-thiocyanomethyl-thiobenzothiazole; C-formals such as 2-hydroxymethyl-2-nitro-1,3-propanediol and 2-bromo-2-nitropropane-1,3-diol; methylene bisthiocyanate; allantoin reaction products; 2-methylisothiazolin-3-one; N-alkyl-1,2-benzisothiazolin-3-ones with 1 to 8 C atoms in the alkyl radical; N-methyl-1,2-benzisothiazolin-3-one; N-butyl-1,2-benzisothiazolin-3-one; 4,5-dichloro-2-n-octylisothiazolin-3-one (DCOIT); 2-n-octylisothiazolin-3-one (OIT); ethanol; formic acid; propan-2-ol; peracetic acid; L-(+)-lactic acid; symclosene (trichloroisocyanuric acid); chloroxylenol (4-chloro-3,5-dimethylphenol); phenoxy-ethanol; nitromethylidyntrimethanol; tosychloramide sodium salt (Chloramine T); dimethyldithiocarbamate potassium salt; benzoic acid sodium salt; o-phthalaldehyde; hydroxy-2-pyridone; 2,6-dimethyl-1,3-dioxan-4-yl acetate; 4,5-dichloro-3H-1,2-dithiol-3-one; 2-butanone peroxide; 2,4-dichlorobenzyl alcohol; 4-(2-nitrobutyl)morpholine; N-(3-aminopropyl)-N-dodecylpropane-1,3-diamine; 2-bromo-1-(4-hydroxyphenyl)ethan-1-one; 2,2′-dithiobis[N-methylbenzamide]; 1,2-benzisothiazol-3(2H)-one; 2-methyl-2H-isothiazol-3-one; troclosene-sodium; sodium dichloroisocyanurate dihydrate; bis(trichloromethyl) sulfone; N,N′-methylenebismorpholine; methylene dithiocyanate; (ethylenedioxy)dimethanol; sodium 2,4,6-trichlorophenolate; pyridine-2-thiol 1-oxide sodium salt; methenamine-3-chloroallyl chloride; 2,2′,2″-(hexahydro-1,3,5-triazin-1,3,5-triyl)triethanol; tetra-1,3,4,6-tetrakis(hydroxymethyl)imidazole[4,5-d]imidazole-2,5(1H,3H)-dione; 1,3-bis(hydroxymethyl)-5,5-dimethylimidazolidine-2,4-dione; (2-bromo-2-nitrovinyl)benzene; didecyldimethylammonium chloride; prometryne; sulfur dioxide; calcium dihexa-2,4-dienoate; iodine; sodium hydrogen sulfite; sodium bromide; sodium hypochlorite; disodium disulfite; hydrogen peroxide; 7a-ethyldihydro-1H,3H,5H-oxazolo[3,4-c]oxazole; sodium sulfite; silver chloride; lignin; boric acid; potassium sulfite; sodium hydrogen 2,2′-methylenebis[4-chlorophenolate]; 2,2-dibromo-2-cyanoacetamide; disodium octaborate tetrahydrate; ammonium bromide; pyrithione-zinc; dodecylguanidine monohydrochloride; potassium 2-biphenylate; (benzyloxy)methanol; sodium p-chloro-m-cresolate; dipotassium disulfite; D-gluconic acid compound with N,N″-bis(4-chlorophenyl)-3,12-diimino-2,4,11,13-tetraazatetradecanediamidine (2:1); p-[(diiodomethyl)sulfonyl]toluene; (benzothiazol-2-ylthio)methyl thioxyanate; potassium (E,E)-hexa-2,4-dienoate; α,α′,α″-trimethyl-1,3,5-triazine-1,3,5(2H,4H,6H)-triethanol; 2-octyl-2H-isothiazol-3-one; bromochloro-5,5-dimethylimidazolidine-2,4-dione; 2-bromo-2-(bromomethyl)pentanonitrile; 4,4-dimethyloxazolidine; 3-iodo-2-propynylbutyl carbamate; tetrakis(hydroxymethyl)phosphonium sulfate (2:1); 4,5-dichloro-2-octyl-2H-isothiazol-3-one; 3,3′-methylenebis[5-methyloxazolidine]; cis-4-[3-(p-tert-butylphenyl)-2-methylpropyl]-2,6-dimethylmorpholine; sodium N-(hydroxymethyl)glycinate; 1,3-didecyl-2-methyl-1H-imidazolium chloride; 1-[1,3-bis-(hydroxymethyl)-2,5-dioxoimidazolidin-4-yl]-1,3-bis(hydroxymethyl)urea; N,N″-bis-(hydroxymethyl)urea; reaction products with 2-(2-butoxyethoxy)ethanol, ethylene glycol and formaldehyde; 5-chloro-2-(4-chlorophenoxy)phenol; 2-butylbenzene[d]isothiazol-3-one; cis-1-(3-chloroallyl)-3,5,7-triaza-1-azoniaadamantane chloride; 3-benzo(b)thien-2-yl-5,6-dihydro-1,4,2-oxathiazine 4-oxide; reaction product of diisopropanolamine with formaldehyde (1:4); silver-zinc-aluminum-boron phosphate glass/glass oxide (silver- and zinc-containing); cyclohexylhydroxydiazene 1-oxide potassium salt and bis[1-cyclohexyl-1,2-di(hydroxy-κ-K)diazeniumato(2-)]-copper; 4,5-trimethylene-2-methylisothiazolin-3-one; zinc pyrithione; IPBC; triclosan and formaldehyde-releasing substances, for example N-formals such as tetramethylolacetylenediurea; N,N′-dimethylolurea; N-methylolurea; dimethyloldimethylhydantoin; N-methylolchloroacetamide; reaction products of allantoin; glycol formals such as ethylene glycol formal; butyl diglycol formals and benzyl formal.

Furthermore suitable as biocides (c) are fungicides such as captan, folpet, metam-sodium, nabam, thiabendazole, dazomet, chlorothalonil, carbendazim, propiconazole and TCMTB (2-benzothiazolylthio)methyl thiocyanate); arthropodicides (insecticides and acaricides) such as α-cypermethrin, cyfluthrin, permethrin, fipronil, imidacloprid, thiametoxam, metaflumizone, amitraz and abamectin (avermectin), and helminthicides.

Preferred biocides are chloro-m-cresol. Fungicides such as o-phenylphenol and its sodium or potassium salts, chlorothalonin, propiconazole and tebuconazole, insecticides such as cyfluthrin, fipronil, imidaclaprid, thiametoxam, metaflumizone and amitraz.

The use of insecticides with antiparasitic activity, in particular against fleas and ticks, is preferred since this controls not only the rodent pests, but also parasites, such as fleas, which are transmitted by them.

Examples of bactericides are Proxel® from ICI or Acticide® RS from Thor Chemie, Kathon® MK from Rohm & Haas and Dowicil® from Dow Elanco.

Component (c) preferably comprises one biocide. Component (c) furthermore preferably comprises two or three, in particular two, biocides. Preferred mixtures of biocides are mixtures comprising one or more bactericides and/or one or more fungicides and one or more insecticides, in particular antiparasitic agents.

Furthermore preferred are mixtures according to the invention comprising one or more bactericides, in particular benzoic acid and its sodium salts, methylisothiazolinone, chloromethylisothiazolinone and 1,2-benzisothiazolin-3-one; one or more fungicides, in particular chlorothalonil, propiconazole and o-phenylophenol and its sodium and potassium salts; and one or more insecticides, in particular pyrethroids, such as cyfluthrin and α-cypermethrin, abanectin, fipronil, neonicotinoids such as imidacloprid and thiametoxam, and organophosphorus compounds such as chlorpyrifos.

The biocide component (c) preferably amounts to from 0.1 to 99% by weight, especially preferably from 1 to 97% by weight.

The rodenticides (a), painkillers (b) and biocides (c) are commercially available compounds which are described, for example, in the following publications, which also provide information on their preparation and manufacturers:
(a) The Pesticide Manual, 14th edition, British Crop Production Council, Farnham, 2006 or The Compendium of Pesticide Common Names;
(b) E. Mutschler et al., Arzneimittelwirkungen, 8th edition, Wissenschaftliche Verlagsgesellschaft MBH, Stuttgart 2001;
(c) Official Journal of the European Union, L 307/39 (Nov. 24, 2003)

Preferred mixtures (M) according to the invention are those which comprise one of the following combinations of components (a), (b) and (c) according to the invention:

| | | |
|---|---|---|
| (M1) | (a6) + (b1) | +one biocide from group (c) |
| (M2) | (a6) + (b2) | +one biocide from group (c) |
| (M3) | (a6) + (b3) | +one biocide from group (c) |
| (M4) | (a6) + (b4) | +one biocide from group (c) |
| (M5) | (a6) + (b5) | +one biocide from group (c) |
| (M6) | (a6) + (b7) | +one biocide from group (c) |
| (M7) | (a6) + (b15) | +one biocide from group (c) |
| (M8) | (a6) + (b16) | +one biocide from group (c) |
| (M9) | (a6) + (b17) | +one biocide from group (c) |
| (M10) | (a6) + (b20) | +one biocide from group (c) |
| (M11) | (a6) + (b29) | +one biocide from group (c) |
| (M12) | (a6) + (b31) | +one biocide from group (c) |
| (M13) | (a6) + (b32) | +one biocide from group (c) |
| (M14) | (a6) + (b33) | +one biocide from group (c) |
| (M15) | (a6) + (b37) | +one biocide from group (c) |
| (M16) | (a6) + (b38) | +one biocide from group (c) |
| (M17) | (a6) + (b49) | +one biocide from group (c) |
| (M18) | (a6) + (b53) | +one biocide from group (c) |
| (M19) | (a6) + (b54) | +one biocide from group (c) |
| (M20) | (a6) + (b55) | +one biocide from group (c) |
| (M21) | (a6) + (b56) | +one biocide from group (c) |
| (M22) | (a6) + (b57) | +one biocide from group (c) |
| (M23) | (a6) + (b58) | +one biocide from group (c) |
| (M24) | (a6) + (b59) | +one biocide from group (c) |
| (M25) | (a6) + (b60) | +one biocide from group (c) |
| (M26) | (a6) + (b61) | +one biocide from group (c) |
| (M27) | (a6) + (b62) | +one biocide from group (c) |
| (M28) | (a6) + (b63) | +one biocide from group (c) |
| (M29) | (a6) + (b64) | +one biocide from group (c) |
| (M30) | (a6) + (b65) | +one biocide from group (c) |
| (M31) | (a6) + (b66) | +one biocide from group (c) |
| (M32) | (a6) + (b67) | +one biocide from group (c) |
| (M33) | (a6) + (b68) | +one biocide from group (c) |
| (M34) | (a6) + (b69) | +one biocide from group (c) |
| (M35) | (a6) + (b70) | +one biocide from group (c) |
| (M36) | (a6) + (b71) | +one biocide from group (c) |
| (M37) | (a6) + (b72) | +one biocide from group (c) |
| (M38) | (a6) + (b73) | +one biocide from group (c) |
| (M39) | (a6) + (b74) | +one biocide from group (c) |
| (M40) | (a6) + (b75) | +one biocide from group (c) |
| (M41) | (a6) + (b76) | +one biocide from group (c) |
| (M42) | (a6) + (b77) | +one biocide from group (c) |
| (M43) | (a8) + (b1) | +one biocide from group (c) |
| (M44) | (a8) + (b2) | +one biocide from group (c) |
| (M45) | (a8) + (b3) | +one biocide from group (c) |
| (M46) | (a8) + (b4) | +one biocide from group (c) |
| (M47) | (a8) + (b5) | +one biocide from group (c) |
| (M48) | (a8) + (b7) | +one biocide from group (c) |
| (M49) | (a8) + (b15) | +one biocide from group (c) |
| (M59) | (a8) + (b16) | +one biocide from group (c) |
| (M51) | (a8) + (b17) | +one biocide from group (c) |
| (M52) | (a8) + (b20) | +one biocide from group (c) |
| (M53) | (a8) + (b29) | +one biocide from group (c) |
| (M54) | (a8) + (b31) | +one biocide from group (c) |
| (M55) | (a8) + (b32) | +one biocide from group (c) |
| (M56) | (a8) + (b33) | +one biocide from group (c) |
| (M57) | (a8) + (b37) | +one biocide from group (c) |
| (M58) | (a8) + (b38) | +one biocide from group (c) |
| (M59) | (a8) + (b49) | +one biocide from group (c) |
| (M60) | (a8) + (b53) | +one biocide from group (c) |
| (M61) | (a8) + (b54) | +one biocide from group (c) |
| (M62) | (a8) + (b55) | +one biocide from group (c) |
| (M63) | (a8) + (b56) | +one biocide from group (c) |
| (M64) | (a8) + (b57) | +one biocide from group (c) |
| (M65) | (a8) + (b58) | +one biocide from group (c) |
| (M66) | (a8) + (b59) | +one biocide from group (c) |
| (M67) | (a8) + (b60) | +one biocide from group (c) |
| (M68) | (a8) + (b61) | +one biocide from group (c) |
| (M69) | (a8) + (b62) | +one biocide from group (c) |
| (M70) | (a8) + (b63) | +one biocide from group (c) |
| (M71) | (a8) + (b64) | +one biocide from group (c) |
| (M72) | (a8) + (b65) | +one biocide from group (c) |
| (M73) | (a8) + (b66) | +one biocide from group (c) |
| (M74) | (a8) + (b67) | +one biocide from group (c) |
| (M75) | (a8) + (b68) | +one biocide from group (c) |
| (M76) | (a8) + (b69) | +one biocide from group (c) |
| (M77) | (a8) + (b70) | +one biocide from group (c) |
| (M78) | (a8) + (b71) | +one biocide from group (c) |
| (M79) | (a8) + (b72) | +one biocide from group (c) |
| (M80) | (a8) + (b73) | +one biocide from group (c) |
| (M81) | (a8) + (b74) | +one biocide from group (c) |
| (M82) | (a8) + (b75) | +one biocide from group (c) |
| (M83) | (a8) + (b76) | +one biocide from group (c) |
| (M84) | (a8) + (b77) | +one biocide from group (c) |
| (M85) | (a18) + (b1) | +one biocide from group (c) |
| (M86) | (a18) + (b2) | +one biocide from group (c) |
| (M87) | (a18) + (b3) | +one biocide from group (c) |
| (M88) | (a18) + (b4) | +one biocide from group (c) |
| (M89) | (a18) + (b5) | +one biocide from group (c) |
| (M90) | (a18) + (b7) | +one biocide from group (c) |
| (M91) | (a18) + (b15) | +one biocide from group (c) |
| (M92) | (a18) + (b16) | +one biocide from group (c) |
| (M93) | (a18) + (b17) | +one biocide from group (c) |
| (M94) | (a18) + (b20) | +one biocide from group (c) |
| (M95) | (a18) + (b29) | +one biocide from group (c) |
| (M96) | (a18) + (b31) | +one biocide from group (c) |
| (M97) | (a18) + (b32) | +one biocide from group (c) |
| (M98) | (a18) + (b33) | +one biocide from group (c) |
| (M99) | (a18) + (b37) | +one biocide from group (c) |
| (M100) | (a18) + (b38) | +one biocide from group (c) |
| (M101) | (a18) + (b49) | +one biocide from group (c) |
| (M102) | (a18) + (b53) | +one biocide from group (c) |
| (M103) | (a18) + (b54) | +one biocide from group (c) |
| (M104) | (a18) + (b55) | +one biocide from group (c) |
| (M105) | (a18) + (b56) | +one biocide from group (c) |
| (M106) | (a18) + (b57) | +one biocide from group (c) |
| (M107) | (a18) + (b58) | +one biocide from group (c) |
| (M108) | (a18) + (b59) | +one biocide from group (c) |
| (M109) | (a18) + (b60) | +one biocide from group (c) |
| (M110) | (a18) + (b61) | +one biocide from group (c) |
| (M111) | (a18) + (b62) | +one biocide from group (c) |
| (M112) | (a18) + (b63) | +one biocide from group (c) |
| (M113) | (a18) + (b64) | +one biocide from group (c) |
| (M114) | (a18) + (b65) | +one biocide from group (c) |
| (M115) | (a18) + (b66) | +one biocide from group (c) |
| (M116) | (a18) + (b67) | +one biocide from group (c) |
| (M117) | (a18) + (b68) | +one biocide from group (c) |
| (M118) | (a18) + (b69) | +one biocide from group (c) |
| (M119) | (a18) + (b70) | +one biocide from group (c) |
| (M120) | (a18) + (b71) | +one biocide from group (c) |
| (M121) | (a18) + (b72) | +one biocide from group (c) |
| (M122) | (a18) + (b73) | +one biocide from group (c) |
| (M123) | (a18) + (b74) | +one biocide from group (c) |
| (M124) | (a18) + (b75) | +one biocide from group (c) |
| (M125) | (a18) + (b76) | +one biocide from group (c) |
| (M126) | (a18) + (b77) | +one biocide from group (c) |
| (M127) | (a36) + (b1) | +one biocide from group (c) |
| (M128) | (a36) + (b2) | +one biocide from group (c) |
| (M129) | (a36) + (b3) | +one biocide from group (c) |
| (M130) | (a36) + (b4) | +one biocide from group (c) |
| (M131) | (a36) + (b5) | +one biocide from group (c) |
| (M132) | (a36) + (b7) | +one biocide from group (c) |
| (M133) | (a36) + (b15) | +one biocide from group (c) |
| (M134) | (a36) + (b16) | +one biocide from group (c) |
| (M135) | (a36) + (b17) | +one biocide from group (c) |
| (M136) | (a36) + (b20) | +one biocide from group (c) |
| (M137) | (a36) + (b29) | +one biocide from group (c) |
| (M138) | (a36) + (b31) | +one biocide from group (c) |
| (M139) | (a36) + (b32) | +one biocide from group (c) |
| (M140) | (a36) + (b33) | +one biocide from group (c) |
| (M141) | (a36) + (b37) | +one biocide from group (c) |
| (M142) | (a36) + (b38) | +one biocide from group (c) |
| (M143) | (a36) + (b49) | +one biocide from group (c) |
| (M144) | (a36) + (b53) | +one biocide from group (c) |
| (M145) | (a36) + (b54) | +one biocide from group (c) |
| (M146) | (a36) + (b55) | +one biocide from group (c) |
| (M147) | (a36) + (b56) | +one biocide from group (c) |
| (M148) | (a36) + (b57) | +one biocide from group (c) |
| (M149) | (a36) + (b58) | +one biocide from group (c) |
| (M150) | (a36) + (b59) | +one biocide from group (c) |
| (M151) | (a36) + (b60) | +one biocide from group (c) |
| (M152) | (a36) + (b61) | +one biocide from group (c) |
| (M153) | (a36) + (b62) | +one biocide from group (c) |
| (M154) | (a36) + (b63) | +one biocide from group (c) |
| (M155) | (a36) + (b64) | +one biocide from group (c) |
| (M156) | (a36) + (b65) | +one biocide from group (c) |
| (M157) | (a36) + (b66) | +one biocide from group (c) |
| (M158) | (a36) + (b67) | +one biocide from group (c) |
| (M159) | (a36) + (b68) | +one biocide from group (c) |
| (M160) | (a36) + (b69) | +one biocide from group (c) |
| (M161) | (a36) + (b70) | +one biocide from group (c) |
| (M162) | (a36) + (b71) | +one biocide from group (c) |
| (M163) | (a36) + (b72) | +one biocide from group (c) |
| (M164) | (a36) + (b73) | +one biocide from group (c) |
| (M165) | (a36) + (b74) | +one biocide from group (c) |
| (M166) | (a36) + (b75) | +one biocide from group (c) |
| (M167) | (a36) + (b76) | +one biocide from group (c) |
| (M168) | (a36) + (b77) | +one biocide from group (c) |

In a preferred embodiment of the invention, the mixture according to the invention consists of the rodenticide, painkiller and biocide component. In this form, it is generally used as what is known as a premix for the preparation of rodenticidal bait formulations according to the invention.

In a further preferred embodiment of the invention, the mixture according to the invention takes the form of a rodenticidal bait formulation which, in addition to the rodenticide, painkiller and biocide components, comprises at least one bait and, if appropriate, further adjuvants and/or additives.

The present invention therefore also relates to a rodenticidal bait formulation comprising
(A) 0.001 to 30% by weight (based on the total of (A), (B) and (C)) of a mixture comprising
   a) 0.0015 to 99.89% by weight (based on the total of (a), (b) and (c)) of at least one rodenticide,
   b) 10⁻⁸ to 95% by weight (based on the total of (a), (b) and (c)) of at least one painkiller,
   c) 0.01 to 99.9984% by weight (based on the total of (a), (b) and (c));
(B) 0.5 to 99.999% by weight (based on the total of (A), (B) and (C)) of at least one bait material, and
(C) 0 to 94.999% by weight (based on the total of (A), (B) and (C)) of one or more adjuvants.

The formulations according to the invention preferably comprise from 10 to 99% by weight (based on the total of (A), (B) and (C)), especially preferably from 20 to 90% by weight, of the bait material.

Bait materials which are generally used are vegetable or animal foodstuffs and feed stuffs. Suitable examples are coarse cereal meals, cereal grains, flaked cereals or cereal meals (for example of oats, wheat, barley, maize, soya, rice), flaked coconut, ground coconut, sugar syrups (for example obtained by hydrolyzing starch (glucose syrup), invert sugar syrup, beet sugar syrup, maple syrup), sugars (for example sucrose, lactose, fructose, glucose), grated nuts, ground nuts (for example hazelnut, walnut, almond), vegetable fat/oils (for example rapeseed oil, soya fat, sunflower oil, cocoa butter, peanut oil, peanut butter, corn oil), animal fats/oils (butter, lard, fish oil), proteins (for example dried skimmed milk, dried egg, protein hydrolysates) and minerals (for example common salt).

Preferred are vegetable foodstuffs such as oatmeal, flaked oats, wheat kernels, coarse wheat meals, wheat flour, maize meal, flaked coconut, ground coconut, glucose syrup, maple syrup, beet sugar syrup, sucrose, glucose, ground hazelnuts, ground walnuts, almond, rapeseed oil, soya fat, peanut oil, corn oil; animal fats such as butter; proteins such as, for example, dried egg and dried skimmed milk.

Especially preferred are vegetable foodstuffs such as oatmeal, maize meal, flaked coconut, ground coconut, glucose syrup, maple syrup, sucrose, ground hazelnuts, soya fat, peanut oil, peanut butter and proteins such as dried skimmed milk.

The formulations according to the invention comprise one or more of the abovementioned bait materials.

It is preferred to employ two different bait materials in the formulations according to the invention.

In addition to the abovementioned feedstuffs, a preferred embodiment of the bait also comprises one or more attractants. For the purposes of the invention, an attractant is a substance (or substance mixture) which is a phagostimulant or which attracts the attention of the rodent pest to the bait without being a feedstuff proper in another way, in particular by odor (for example as a sexual attractant). Examples of attractants are pheromones, yeast, ground crustaceans, fecal matter, berries, chocolate, fish meal, meat, black pepper and flavor enhancers such as glutamates, in particular sodium glutamate and disodium glutamate.

In general, the attractants amount to from 0 to 10% by weight, preferably from 0 to 1% by weight, based on the total formulation.

Further customary additives (C) comprise colorants, bittering agents, flow agents, binders, agents for improving weather resistance, and antioxidants.

Colorants are frequently added, and the bait formulation is thereby clearly logged as not for consumption, in order to avoid ingestion by mistake by humans or non-target animals. In particular, blue colorants serve to deter birds. However, they may also serve to detect the consumption of the bait in the rodent pests' feces or vomit.

Bittering agents serve to avoid incidental consumption by humans. Especially preferred is denatonium benzoate, which, in a suitable concentration (in general 1 to 200 ppm, in particular 5 to 20 ppm), has a most unpleasant taste for humans, but not for rodents.

Flow agents and binders are added as a function of the bait formulation type. Binders are capable of fixing the mixture according to the invention onto the surface of the bait component (for example cereal grains) or—in the case of pastes for example—impart structure and coherence. Flow agents such as mineral earths and aluminosilicates facilitate extrudation and they are therefore frequently used in pellets and extruded blocks.

Suitable agents for improving weather resistance are, for example, paraffin waxes.

Examples of suitable antioxidants are t-butylhydroquinone (TBHQ), butylated hydroxytoluenes and butylated hydroxyanisoles, preferably in an amount of from 10 ppm to 20 000 ppm.

In general, the adjuvants (C) used will depend on the nature of the bait formulation used.

Preferred bait formulations are food baits, in particular seed cereal baits and suitable treatment agents, pellets (die-formed articles), wax-coated pellets, molten-wax blocks, compressed or extruded wax blocks, pastes, gels, granules and foams.

Feed baits frequently consist of cereal which may be present in different fine forms, for example in the form of grains or else as a more or less finely ground meal.

The advantage of a bait in the form of a meal is that it is difficult to carry away and store by the animals, but is not easy to handle (dust) and spoils rapidly. The disadvantage of intact grains is that they may also be ingested by non-target animals, for example birds, but under certain circumstances also humans.

The mixture according to the invention and further adjuvants may be applied to the surface of the feedstuffs, in particular in conjunction with binders. Preferred is therefore the treatment of such feedstuffs with the mixture according to the invention in the manner of a seed treatment. Thus, for example, WO 2007/057393 discloses a rodenticide formulation comprising
at least one rodenticide;
at least one polyol;
an adhesive, and, if appropriate,
a monosaccharide and/or a disaccharide and/or an oligosaccharide.

Suitable treatments according to the invention comprise from 0.01% by weight to 30% by weight, preferably from 0.01 to 1% by weight, especially preferably from 0.02 to 0.2% by weight, of a mixture according to the invention of components (a), (b) and (c).

The polyol content is from 1% by weight to 50% by weight, preferably 1 to 20% by weight.

The adhesive content is from 1% by weight to 30% by weight, preferably from 1 to 10% by weight, especially preferably from 1 to 5% by weight.

The monosaccharide and/or disaccharide and/or oligosaccharide content is from 10% by weight to 50.0% by weight, preferably from 10.0 to 35.0% by weight, especially preferably from 15.0 to 25.0% by weight.

Suitable polyols are glycol, polyethylene glycol, glycerol, propylene glycol, dipropylene glycol, preferably glycerol.

The use of mono- and disaccharides is preferred. The use of disaccharides is very especially preferred.

Suitable monosaccharides are glucose, fructose, galactose, preferably fructose.

Suitable disaccharides are sucrose, maltose, lactose, preferably sucrose (for example in pure form or as molasses, beet sugar).

A suitable oligosaccharide is starch.

Suitable adhesives are ethylene oxide/propylene oxide copolymers, polyvinyl alcohol (for example Mowiol®4-98, Clariant, Rhodoviol®60-20, Rhone-Poulence), polyvinylpyrrolidone (Sokalan® HP 50, BASF, Kollidon® 25, BASF, Luvitec® K80, BASF Agrimer® A, ISP Global Techn), polyacrylates (for example Sokalan® PA 110 S, BASF), polymethyl methacrylates, water-soluble polyolefin derivatives such as polybutene derivatives, polyethylene oxides (for example polyethers) or polyisobutyl derivatives (for example copolymers of polyolefins and maleic anhydride derivatives (for example Densodrin BA® by BASF), polystyrene derivatives (for example copolymers of styrene and maleic anhydride derivatives or copolymers of styrene and acrylic acid derivatives, or styrene/butadiene-based latex copolymers, obtainable, for example, under the name Semkote E-125, Uniqema) and polyethyleneamines, polyethyleneamides, polyethyleneimines (for example Lupasol® BASF, Polymin®, BASF), polyurethanes (Semkote E-105, Uniqema), polyvinyl acetate, tylose of and polyethylene wax (for example commercially available under the name Poligen® WE 7 BASF, preferably

ethylene oxide/propylene oxide copolymers, polyacrylates (for example Sokalan® PA 110 S, BASF), polymethyl methacrylates, water-soluble polyolefin derivatives such as polybutene derivatives, polyethylene oxides (for example polyethers) or polyisobutyl derivatives (for example copolymers of polyolefins and maleic anhydride derivatives (for example Densodrin BA® by BASF), polystyrene derivatives (for example copolymers of styrene and maleic anhydride derivatives or copolymers of styrene and acrylic acid derivatives, or styrene/butadiene-based latex copolymers, obtainable, for example, under the name Semkote E-125, Uniqema), polyethyleneamines, polyethyleneamides, polyethyleneimines (for example Lupasol® BASF, Polymin®, BASF), polyurethanes (Semkote E-105, Uniqema), polyvinyl acetate, and polyethylene wax (for example commercially available under the name Poligen® WE 7 BASF); especially preferably

ethylene oxide/propylene oxide copolymers, polyacrylates (for example Sokalan® PA 110 S, BASF), polymethyl methacrylates, polystyrene derivatives (for example copolymers of styrene and maleic anhydride derivatives or copolymers of styrene and acrylic acid derivatives, or styrene/butadiene-based latex copolymers, obtainable, for example, under the name Semkote E-125, Uniqema) and polyethylene wax (for example commercially available under the name Poligen® WE 7 BASF);

Moreover, the formulations according to the invention may optionally comprise yet further adjuvants such as, for example, surfactants (such as wetting agents, adhesives and dispersants), antifoams, thickeners and colorants.

Examples of surfactants are alkali metal, alkaline-earth metal, ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ether, tributylphenyl polyglycol ether, tristerylphenyl polyglycol ether, alkylaryl polyether alcohols, alcohol and fatty alcohol/ethylene oxide condensates, ethoxylated castor oil, polyoxyethylene alkyl ether, ethoxylated polyoxypropylene, lauryl alcohol polyglycol ether acetal, sorbitol esters, ligninsulfite waste liquors and methylcellulose.

Examples of thickeners (i.e. compounds which impart a pseudo-plastic flow behavior to the formulation, i.e. high viscosity in the state of rest and low viscosity in the state of motion) are, for example, polysaccharides or organic layer minerals such as xanthan gum (Kelzan® from Kelco), Rhodopol® 23 (Rhone Poulenc) or Veegum® (from R.T. Vanderbilt) or Attaclay® (from Engelhardt).

Examples of antifoams are silicone emulsions (such as, for example, Silikon® SRE, from Wacker or Rhodorsil® from Rhodia), long-chain alcohols, fatty acids, organofluorine compounds and their mixtures.

Colorants which are suitable are all the colorants which are conventionally used for such purposes. Both pigments, which are sparingly soluble in water, and dyes, which are soluble in water, may be used. Examples which may be mentioned are the colorants known by the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108 and their dyes such as, for example, Disperse Blue 69-0007, compounds available from BASF.

The treatment can be carried out by methods known to the skilled worker (for example by spraying or immersing/incubating the cereal grains in, or with, a formulation according to the invention, if appropriate using a suitable device such as a continuously or batchwise-operating seed dresser). In doing so, the formulation may preferably be diluted with up to 7.5 g of water/kg of cereal grains. The treated grains may optionally be dried.

Furthermore preferred as bait formulation are what are known as pellets (die-formed articles). Such pellets comprise the mixture (A) according to the invention in a mixture with optionally powdered or ground feedstuffs (B), in particular cereal and thickeners, and other adjuvants (C). Pellets are usually prepared by compressing, extrusion and subsequent drying.

The pellet size varies as a function of the target animals. Frequently, pellets are prepared in the form of cylinders of diameter 3 to 5 mm and a length of 5 to 10 mm.

In general, the mixture (A) according to the invention amounts to from 0.001 to 30% by weight in the pellets.

To increase the weather resistance of pellets, paraffin wax is added in one embodiment of the invention, which, however, reduces the palatability of the pellets for the rodent pests.

A further preferred bait formulation are wax block formulations which comprise a mixture of feedstuffs, typically cereal grains, coarse cereal meals or cereal powders (B) of component (A) according to the invention, if appropriate adjuvants (C) and paraffin wax. Wax block formulations have the advantage that their weather resistance is good; however, at the expense of the palatability for the rodent pests. Wax block formulations are usually prepared by casting, extruding or compressing, the wax content in the last-mentioned methods being lower, which—with a similarly good weather resistance—increases palatability. Wax block formulations may be prepared in many shapes which allow them for example to be hung up or fastened in a bait station. In a preferred embodiment, the wax blocks comprise a multiplicity of corners because the animals prefer to gnaw at corners.

A further preferred bait formulation are granules which, besides component (A) according to the invention, comprise a typically comminuted, for example ground feedstuff (R) and, if appropriate, further adjuvant (C), a binder. The preparation of granules is described for example in EP-A 0 771 393.

A multiplicity of methods may be used for preparing the bait in granule form, for example mechanical stirring or fluidized-bed granulation. It is preferred to use the mechanical stirring method. Examples of machines which may be employed in such a mechanical stirring method are:

Vat granulators, rotary drum granulators, twin-arm mixers, plowshare mixers (Lodige), high-shear granulators and conveyor mixers. Although the process steps may vary as a function of specifically selected methods, a typical process includes the following steps:
(a) The dry powder components (for example ground cereals and, if appropriate, attractants are carefully mixed with one another in a suitable mixer in order to form a premix. Typical mixing times are in the order of 1 to 10 minutes, depending on the machines used. The mixer used for this process step may also be the means on which granulation is achieved.
(b) Component (A) and, if appropriate, further adjuvants (C), both preferably in the form of a liquid concentrate, are added to the premix described in (a), and the mixture is mixed until homogeneous. Typically, the concentrate is sprayed onto the premix in order to support the generation of a uniform distribution. Typical mixing times are in the order of from 10 to 30 minutes.
(c) An aqueous solution of the film-forming binder, which solution comprises a preservative, if appropriate, is sprayed onto the mixture described in (b) while the mixture is being mixed. As an alternative, the solution of the film-forming binder may be mixed with the active substance concentrate, and the resulting, sole liquid component may be sprayed onto the powder premix.
(d) The wet composition is mixed for the required period of time until granulation has been achieved and the required particle size range has been generated, which would typically be in the order of from 1 to 5 minutes.
(e) The granule mass is then dried to a given degree of moisture, either in the granulator or in a separate device (for example a fluidized-bed drier). A typical moisture content would be in the order of from 1 to 15%. The drying times may vary and depend on the original moisture content, drying temperature, the air flow and the like. A typical drying time is 80 to 100° C., and drying times are in the order of 30 minutes.
(f) The granules are sorted into the desired particle size range (for example by screening).

A furthermore preferred bait formulation are gels (see, for example, WO 03/094612). Preferably, such gels comprise
water as dispersant;
thickener;
component (A) according to the invention;
feedstuffs;
   in a preferred embodiment, the gels according to the invention additionally also comprise one or more of the following components:
base;
humectant;
oxidation stabilizers;
colorants;
bittering agents;
further additives.

Thickeners which are used are organic and inorganic macromolecules. Organic macromolecules which may be mentioned are cellulose derivatives, for example hydroxypropylcellulose, hydroxyethylcellulose, methylcellulose, carboxymethyl-cellulose-sodium, hydroxypropylmethylcellulose, hydroxyethylmethylcellulose, hydroxyethylpropylcellulose such as xanthans, alginates, carrageenan, agar-agar, polyvinyl alcohols, polyvinylpyrrolidone, polyacrylic acid and polymethacrylic acid. Inorganic macromolecules (inorganic gel formers) which may be mentioned is highly-dispersed silica and bentonites (for example Rudolf Voigt, Pharmazeutische Technologie [Pharmaceutical technology], pages 362 to 385, Ulstein Mosby).

Bases which are employed are, for example, alkali metal hydroxides, alkaline earth metal hydroxides and amino derivatives, such as potassium hydroxide, sodium hydroxide, triethanolamine or ammonium hydroxide solution. Bases may be employed for adjusting the pH.

Humectants which are employed are, for example, polyols such as glycerol, propylene glycol, ethylene glycol, sugar alcohols and polypropylene glycols. Humectants prevent the gel from drying out so that it remains elastic and is not scattered when taken up by the rodents.

Preferred are glycerol, propylene glycol, polypropylene glycol 200, propylene glycol 300, polypropylene glycol 400, sorbitol, mannitol and xylitol.

Oxidation stabilizers which may be mentioned are butylhydroxytoluene, butylhydroxyanisole, tocopherols (for example vitamin E) or ascorbic acid and its derivatives (for example ascorbic acid palmitate, sodium ascorbate). The foodstuffs and feedstuffs can be stabilized by adding oxidation stabilizers.

Especially preferred are butylhydroxytoluene, vitamin E, ascorbic acid.

Colorants which may be mentioned are blue, green and red pigments and blue, green and red soluble dyes (see Colour Index, Fourth Edition: Hue blue, red, green), with blue colorants being preferred for deterring birds. A gel according to the invention may be colored by colorants for warning purposes.

Suitable colorants which are also approved for coloring cosmetics are preferred (cf., for example, Otterstätter, Die Färbung von Lebensmitteln, Arzneimitteln, Kosmetika [Coloring foodstuffs, pharmaceuticals, cosmetics], Behr's Verlag, 2nd edition, pages 52 to 57).

It is preferred to use pigments.

Additives which may be used are, for example, bittering agents such as denatonium benzoate and natural and synthetic aroma chemicals (see, for example, product list of Hamann & Reimer, Holzminden).

Gels according to the invention comprise the following, expressed in percent by weight:
0.3 to 15% thickener,
0.0005 to 1.0% of component (A) according to the invention,
10 to 80% feedstuffs,
if appropriate 0.5 to 8% base,
if appropriate 3 to 25% humectant,
if appropriate 0.01 to 2% oxidation stabilizers,
if appropriate 0.001 to 0.2% colorants,
if appropriate 0.001 to 0.02% additives
and the remaining percent by weight water.

The viscosity of the gels can be varied within wide limits and is in general 5000 to 100 000 mPas. A preferred viscosity range is 8000 to 60 000 mPas, an especially preferred viscosity range is 10 000 to 50 000 mPas. The viscosity is measured using a rotary viscometer (Haake) at a shear rate of D=10 [1(sec], measured using a plate-cone measuring system)(4°). As a result of the heterogeneity of the gels, the data recorded vary. The viscosity may be adjusted by varying the nature and the percentage by weight of the components, in particular of the thickener and of the bait material.

The pH of the gels according to the invention can be varied within wide limits and is generally between pH 2 and pH 10. A preferred pH range is between pH 3 and pH 8.

A further preferred type of bait formulation are high-resistant foams or flexible foams, preferably flexible foams.

High-resistant foams which can be employed in accordance with the invention are described for example in FR-PS 2 676 888 and U.S. Pat. No. 4,190,734. Flexible foams which can be employed in accordance with the invention are described for example in GB-PS 1 053 088 and GB 1 274 442.

Preferred foams are the flexible foams described in DE-A 44 44 261. Such flexible foams comprise
a component (A) according to the invention,
hydrophilic polymers with a mean molecular weight of from 2000 to 60 000 (determined by gel permeation chromatography (GPC)) from the series of the long-chain polyurethanes, polyesters, polyester polyols, polystyrenes, polybutadienes, maleic acid polymers, each of which is modified in the polymer chain by carboxyl or amino groups,
long-chain aliphatic C₆-C₂₂-fatty acids such as palmitic acid, dodecanoic acid and stearic acid, or their alkali metal, alkaline earth metal and ammonium salts,
and, if appropriate, further adjuvants from the series consisting of colorants, emulsifiers, solvents, attractants and feedstuffs.

The polymers used are described for example in H. Kittel, Lehrbuch der Lacke and Beschichtungen [Textbook of paints and coatings], Volume IV, pages 76 to 306, Verlag W.A. Colomb (1096) or in the same textbook, edition (1976), Volume IV, pages 328 to 358 as binders for paints.

Polymers which may be used in accordance with the invention are physically drying binders, for example those whose binders are based on a fully reacted, linear polyurethane of (i) a polyester polyol, (ii) a chain extender and (iii) a diisocyanate and (iv) a hydroxycarboxylic acid.

Suitable polyester polyols (i) for the preparation of such polyurethanes are, for example, adipic acid, alkanediol, polyester diols of the molecular weight range from 600 to 3000. The alkanediols are, for example, butane-1,4-diol, hexane-1,6-diol, neopentyl glycol or mixtures of such glycols. Suitable chain extenders (ii) are, for example, diols of the type employed for the preparation of the polyester diols, and also diamines such as hexamethylenediamine or isophoronediamine. Examples of suitable diisocyanates (iii) are 4,4′-diphenylmethane diisocyanate, isophorone diisocyanate or hexamethylene diisocyanate. The polyurethanes are prepared in the manner known per se by reacting the starting materials, with an equivalent ratio of isocyanate groups to isocyanate groups of reactive groups of from 0.9:1 to 1.1:1 being maintained.

Oxidatively drying binders may also be used. Such binders which may be mentioned are those based on polybutadiene, styrene and maleic anhydride and having ionic groups, as they are described in the applications EP-OS 0 170 184 and EP-OS 0 270 795.

The hydrophilic polymers have a mean molecular weight of from 2000 to 60 000 g/mol, preferably of from 2500 to 25 000 g/mol. They are present in the finished formulation in a concentration of from 2.5 to 40, preferably 2.5 to 10, % by weight based on the weight of the total formulation.

The systems thus prepared are premixes. As a rule, they must be diluted with water in amounts of from 0 to 80% before being applied.

The flexible foams can be prepared in a manner known per se by stirring or shaking. Another possibility is the preparation in situ during application, using blowing agents.

Blowing agents for the preparation of the formulations according to the invention which may be mentioned are CO₂N₂O, lower alkanes such as propane or n-butane, iso-butane, halogen-containing lower alkanes and low-boiling ethers such as dimethyl ether, and mixtures of said blowing agents.

In a further preferred embodiment of the invention, the bait formulation according to the invention comprises a particulate mixture which, besides particles comprising a feed stuff and the mixture (A) according to the invention, also comprises particles which comprise a feedstuff with a flavor which differs from that of the first feedstuff and which differ from the first-mentioned particles in terms of size, shape, surface texture, internal texture, color, density and/or content. For example, the non-rodenticidal particles are present in an amount of from 2.5 to 10% by weight (based on the total formulation). The non-rodenticidal particles are preferably based on cereals and preferably comprise at least one further attractant from the group consisting of chocolate, dried and ground crustaceans, yeast, fecal matter, fish meal, meat and berries.

Examples of such particulate formulations are described in WO 2007/031796.

The bait formulations according to the invention are suitable for controlling rodent pests.

In accordance with the invention, the term “rodent pests” as well as rodents also comprises further harmful vertebrates, with the exception of humans.

Rodent pests to be controlled are preferably rodents from the order Rodentia, preferably from the family Musidae, in particular Murinae. Particular attention is to be given to the genera Rattus and Mus, Microtus, furthermore also, inter alia, Apodemus, Microtus, Arvicola and Clethrionomys, in particular the species Rattus norvegicus, Rattus rattus, Rattus argentiventer, Rattus exulans, Mus sp. Arvicola terrestris, Microtus arvalis, Microtus pennsylvanicus, Tatera indica, Peromyscus leucopus, Peromyscus maniculatus, Mastomys natalensis, Sigmodon hispidus, Arvicanthis niloticus, Bandicota bengalensis, Bandicota indica, Nesokia indica, Meriones hurrinanae and Millardia meltada. Very especially attention is to be given to the representatives of the genera Rattus and Mus, for example R. rattus, R. norvegicus, M. musculus, preferably.

In addition, the formulations according to the invention are also suitable for controlling other harmful vertebrates, for example opossums (vulpes vulpes), and American opossums (Didelphidae), brushtail possums (Trichosurus, in particular the common brushtail possum (Trichosurus vulpecula), nutria (Myocastor coypus), rabbit (i.e. suitable genera from the subfamily Leporinae and raccoons, in particular Procyon Cofor.

The invention therefore also relates to a method of controlling rodent pests, wherein a bait formulation according to the invention is applied in the habitat of the harmful vertebrates.

The invention furthermore relates to the use of a bait formulation according to the invention for controlling rodent pests.

The bait formulation according to the invention is suitable for application in rooms, for example cellars, stores, pantries, animal houses, or in gutters and in the open, for example in runs of the rodent pests, or in holes in which they dwell.

In one embodiment of the invention, the bait formulation according of the invention is applied in a bait box. Such bait boxes are described for example in U.S. Pat. No. 3,750,326, U.S. Pat. No. 4,349,982, DE-A 195 01 892, WO 02/102147, DE-A 10 2004 022 105 and DE-A 10 2004 022 103.

Individual features of the mixtures and bait formulations according to the invention can be combined as desired. Such combinations are also subject matter of the invention. Dosage instructions represent the general usage, but larger or smaller amounts may also be used in individual cases.

Subject matter of the invention is therefore also a bait box comprising a bait formulation according to the invention.

The invention is illustrated in greater detail by the examples.

### EXAMPLES

### Example 1

Formulation type: wax block prepared by compressing a homogenized mixture consisting of:

| | |
|---|---|
| Zinc phosphate | 2% |
| Paracetamol | 1 × 10⁻³% |
| Tramol | 1 × 10⁻⁵% |
| Orthophenyl phosphate | 0.04% |
| Cyfluthrin | 0.001 |
| Acticide MBS ® | 0.1% |
| Wheat flour | 27% |
| Coarse wheat meal | 32% |
| Maize meal | 3% |
| Millet | 5% |
| Wax powder | to 100% |
| Sodium chloride | 0.5% |
| Denatonium benzoate | 0.002% |
| Disperse Blue | 0.3% |
| Monosodium glutamate | 0.5% |
| Acticide ® MBS | (mixture of methyl-4-isothiazoline |
| | and 1,2-benziso-thiazolin-3-one, Thor |
| | GmbH, Speyer, Germany) |

### Example 2

Formulation type: wax block prepared by compressing a homogenized mixture consisting of

| | |
|---|---|
| Flocoumafen | 0.005% |
| (1% of 0.5% Storm ® mastermix) | |
| Paracetamol | 1 × 10⁻³% |
| Tramol | 1 × 10⁻⁶% |
| Orthophenyl phosphate | 0.04% |
| Cyfluthrin | 0.001 |
| Acticide MBS ® | 0.1% |
| Wheat flour | 27% |
| Coarse wheat meal | 32% |
| Maize meal | 3% |
| Millet | 5% |
| Wax powder | to 100% |
| Sodium chloride | 0.5% |
| Denatonium benzoate | 0.002% |
| Disperse Blue | 0.3% |
| Monosodium glutamate | 0.5% |

Storm ® = commercially available flocoumafen formulation, comprising 5 g/kg flocoumafen, BASF Aktiengesellschaft, Ludwigshafen, Germany)

### Example 3

Formulation type: pellets—prepared by extruding a homogeneous mixture consisting of:

| | |
|---|---|
| Flocoumafen | 0.005% |
| (1% of 0.5% Storm ® mastermix) | |
| Paracetamol | 1 × 10⁻³% |
| Methadone | 1 × 10⁻⁶% |
| Orthophenyl phosphate | 0.04% |
| Cyfluthrin | 0.001 |
| Acticide MBS | 0.1% |
| Wheat flour | to 100% |
| Maize meal | 2% |
| Millet | 10% |
| Sucrose | 4% |
| Sodium chloride | 0.5% |
| Denatonium benzoate | 0.002% |
| Disperse Blue | 0.3% |
| Monosodium glutamate | 0.5% |

### Example 4

### Formulation Type: CB (Cereal Bait)

Premix

| | | |
|---|---|---|
| Flocoumafen (0.5% Storm ® mastermix) | 40 | g |
| Paracetamol | 40 | g |
| Methadone | 0.004 | g |
| Orthophenyl phosphate | 0.02 | g |
| Cyfluthrin | 0.001 | g |
| Acticide MBS | 2 | g |
| Beet sugar | 150 | g |
| Glycerol | 100 | g |
| Poligen WE 7 | 50 | g |
| Fipronil | 0.001 | g |
| Denatonium benzoate | 0.004 | g |
| Disperse Blue | 10 | g |
| Thickener | 100 | g |

(Preswelled xanthan (2%)/water mixture)

### CB Preparation:

In a stirred vessel, the components of the premix are introduced in succession, with stirring, and the mixture is homogenized with stirring at room temperature.

1000 g of wheat grains are introduced into a commercially available continuously or batchwise-operating seed dresser (batch seed dresser Concept ML 2000/Satec). Thereafter, 20 g of the above premix are metered in under mild conditions (approx. 690 rpm) using a roller pump. After a further 30 sec, the dressed grains are transferred into suitable containers.

1-12. (canceled)

13. A rodenticidal mixture comprising
a) 0.0015 to 99.89% by weight of at least one rodenticide,
b) 10⁻¹¹ to 95% by weight of at least one painkiller and
c) 0.01 to 99.9984% by weight of at least one biocide,
where the percentages by weight are based on the total of components (a), (b) and (c).

14. The rodenticidal mixture according to claim 13, comprising a rodenticide from group brodifacoum (a1), bromadiolone (a2), coumachlor (a3), coumafuryl (a4), coumatetralyl (a5), difenacoum (a6), difethialone (a7), flocoumafen (a8) and warfarin (a9), chlorophacinone (a10), diphacinone (a11), pindone (a12), arsenic oxide (a13), phosphorus (a14), potassium arsenite (a15), sodium arsenite (a16), thallium sulfate (a17), zinc phosphide (a18); γ-HCH (a19), HCH (a20), lindan (a21); phosacetim (a22); crimidine (a23); antu (a24); pyrinuron (a25); scilliroside (a26), strychnine (a27); bromethalin (a28), chloralose (a29), α-chlorohydrin (a30), ergocalciferol (a31), fluroroacetamide (a32), flupropadine (a33), norbormide (a34), sodium fluoroacetate (a35) or vitamin D3 (a36).

15. The rodenticidal mixture according to claim 14, comprising a rodenticide from the group consisting of difenacoum, flocoumafen, zinc phosphide and vitamin D3.

16. The rodenticidal mixture according to claim 13, comprising a painkiller selected from the group consisting of morphine (b1), codeine (b2), dihydrocodeine (b3), hydromorphone (b4), oxycodone (b5), hydrocodone (b6), pethidine (b7), levomethadone (b8), levacetylmethadol (b9), piritramide (b10), pentazocine (b11), buprenorphine (b12), nalbuphine (b13), tilidine (b14), tramadol (b15), methadone (b16), acetylsalicylic acid (b17), amides of salicylic acid (b18), salsalate (b19), diflunisal (b20), indometacin (b21), acemetacin (b22), diclofenac (b23), lonazolac (b24), ibuprofen (b25), flurbiprofen (b26), ketoprofen (b27), dexketoprofen (b28), naproxen (b29), tiaprofen acid (b30), piroxicam (b31), tenoxicam (b32), meloxicam (b33), lornoxicam (b34), mefenamic acid (b35), flufenamic acid (b36), paracetamol (b37), phenazone (b38), propyphenazone (b39), metamizole (b40), nabumetone (b41), azapropazone (b42), aceclofenac (b43), oxaceprol (b44), rofecoxib (b45), celecoxib (b46), nefopam (b47), flupirtin (b48), propofol (b49), neuroleptics (b50), fentanyl (b51), sulfentanil (b52), clonidine (b53), phenobarbital (b54), pentobarbital (b55), alprazolam (b56), bromazepam (b57), brotizolam (b58), chlordiazepoxide (b59), clobazam (b60), clonazepam (b61), diazepam (b62), clorazepate (b63), flunitrazepam (b64), flurazepam (b65), loprazolam (b66), lorazepam (b67), lormetazepam (b68), medazepam (b69), midazolam (b70), nitrazepam (b71), nordazepam (b72), oxazepam (b73), prazepam (b74), temazepam (b75), tetrazepam (b76) and triazolam (b77).

17. The rodenticidal mixture according to claim 16, comprising a painkiller selected from the group consisting of morphine, codeine, dihydrocodeine, hydromorphine, oxycodone, pethidine, tramadol, methadone, acetylsalicylic acid, diflunisal, naproxen, proxicam, tenoxicam, meloxicam, paracetamol and phenazone, propotol, clonidine, phenobarbital, pentobarbital, alprazolam (b56), bromazepam (b57), brotizolam (b58), chlordiazepoxide (b59), clobazam (b60), clonazepam (b61), diazepam (b62), clorazepate (b63), flunitrazepam (b64), flurazepam (b65), loprazolam (b66), lorazepam (b67), lormetazepam (b68), medazepam (b69), midazolam (b70), nitrazepam (b71), nordazepam (b72), oxazepam (b73), prazepam (b74), temazepam (b75), tetrazepam (b76) and triazolam (b77).

18. The rodenticidal mixture according to claim 13, wherein the at least one painkiller comprises a mixture of one or more analgesics and one or more sedatives.

19. The rodenticidal mixture according to claim 13, where the biocide is selected from the group consisting of benzyl alcohol; 2,4-dichlorobenzyl alcohol; 2-phenoxyethanol; 2-phenoxyethanol hemiformal; phenyl ethyl alcohol; 5-bromo-5-nitro-1,3-dioxane; bronopol; formaldehyde; dimethyloldimethylhydantoin; glyoxal; glutardialdehyde; sorbic acid; benzoic acid; salicylic acid; p-hydroxybenzoic ester; chloroacetamide; N-methylolchloroacetamide; p-chloro-m-cresol; o-phenylphenol; sodium o-phenylphenolate; 4,4-dimethyl-1,3-oxazolidine; 1,3,5-hexahydrotriazine derivatives; N(C₁₂-C₁₈)-, (C₁₂-C₁₆)- or (C₁₂-C₁₄)-alkyl-N,N-dimethylbenzylammonium chloride; N-di-C₈-C₁₀-alkyldimethylammonium chloride; di-n-decyldimethylammonium chloride; C₁₂-C₁₄-alkyl[(ethylphenyl)methyl]dimethylammonium chloride; [2-[[2-[carboxyethyl)(2-hydroxyethyl)amino]ethyl]amino]-2-oxoethyl]cocoalkyldimethyl hydroxides (internal salts); cetylpyridinium chloride; diguanidine; polybiguanide; chlorhexidine; 1,2-dibromo-2,4-dicyanobutane; 3,5-dichloro-4-hydroxybenzaldehyde; ethylene glycol hemiformal; tetra(hydroxymethyl)phosphonium salts; chlorophen; dichlorophen; 2,2-dibromo-3-nitrilopropionamide; 3-iodo-2-propynyl N-butylcarbamate; methyl N-benzimidazol-2-ylcarbamate; di-N-methyl-2,2′-dithiodibenzamide; 2-thiocyanomethylthiobenzothiazole; 2-hydroxymethyl-2-nitro-1,3-propanediol and 2-bromo-2-nitropropane-1,3-diol; methylene bisthiocyanate; allantoin reaction products; 2-methylisothiazolin-3-one; N-alkyl-1,2-benzisothiazolin-3-ones with 1 to 8 C atoms in the alkyl radical; N-methyl-1,2-benzisothiazolin-3-one; N-butyl-1,2-benzisothiazolin-3-one; 4,5-dichloro-2-n-octylisothiazolin-3-one (DCOIT); 2-n-octylisothiazolin-3-one (OIT); ethanol; formic acid; propan-2-ol; peracetic acid; L-(+)-lactic acid; symclosene (trichloroisocyanuric acid); chloroxylenol (4-chloro-3,5-dimethylphenol); phenoxyethanol; nitromethylidyntrimethanol; tosychloramide sodium salt (Chloramine T); dimethyldithiocarbamate potassium salt; benzoic acid sodium salt; o-phthalaldehyde; hydroxy-2-pyridone; 2,6-dimethyl-1,3-dioxan-4-yl acetate; 4,5-dichloro-3H-1,2-dithiol-3-one; 2-butanone peroxide; 2,4-dichlorobenzyl alcohol; 4-(2-nitrobutyl)morpholine; N-(3-aminopropyl)-N-dodecylpropane-1,3-diamine; 2-bromo-1-(4-hydroxyphenyl)ethan-1-one; 2,2′-dithiobis[N-methylbenzamide]; 1,2-benzisothiazol-3(2H)-one; 2-methyl-2H-isothiazol-3-one; troclosene-sodium; sodium dichloroisocyanurate dihydrate; bis(trichloromethyl) sulfone; N,N′-methylenebismorpholine; methylene dithiocyanate; (ethylenedioxy)dimethanol; sodium 2,4,6-trichlorophenolate; pyridine-2-thiol 1-oxide sodium salt; methenamine-3-chloroallyl chloride; 2,2′,2″-(hexahydro-1,3,5-triazin-1,3,5-triyl)triethanol; tetra-1,3,4,6-tetrakis(hydroxymethyl)imidazole[4,5-d]imidazole-2,5(1H,3H)-dione; 1,3-bis(hydroxymethyl)-5,5-dimethylimidazolidine-2,4-dione; (2-bromo-2-nitrovinyl)benzene; didecyldimethylammonium chloride; prometryne; sulfur dioxide; calcium dihexa-2,4-dienoate; iodine; sodium hydrogen sulfite; sodium bromide; sodium hypochlorite; disodium disulfite; hydrogen peroxide; 7a-ethyldihydro-1H,3H,5H-oxazolo[3,4-c]oxazole; sodium sulfite; silver chloride; lignin; boric acid; potassium sulfite; sodium hydrogen 2,2′-methylenebis[4-chlorophenolate]; 2,2-dibromo-2-cyanoacetamide; disodium octaborate tetrahydrate; ammonium bromide; pyrithione-zinc; dodecylguanidine monohydrochloride; potassium 2-biphenylate; (benzyloxy)methanol; sodium p-chloro-m-cresolate; dipotassium disulfite; D-gluconic acid compound with N,N″-bis(4-chlorophenyl)-3,12-diimino-2,4,11,13-tetraazatetradecanediamidine (2:1); p-[(diiodomethyl)sulfonyl]toluene; (benzothiazol-2-ylthio)methyl thioxyanate; potassium (E,E)-hexa-2,4-dienoate; α,α′,α″-trimethyl-1,3,5-triazine-1,3,5(2H,4H,6H)-triethanol; 2-octyl-2H-isothiazol-3-one; bromochloro-5,5-dimethylimidazolidine-2,4-dione; 2-bromo-2-(bromomethyl)pentanonitrile; 4,4-dimethyloxazolidine; 3-iodo-2-propynylbutyl carbamate; tetrakis(hydroxymethyl)phosphonium sulfate (2:1); 4,5-dichloro-2-octyl-2H-isothiazol-3-one; 3,3′-methylenebis[5-methyloxazolidine]; cis-4-[3-(p-tert-butylphenyl)-2-methylpropyl-2,6-dimethylmorpholine; sodium N-(hydroxymethyl)glycinate; 1,3-didecyl-2-methyl-1H-imidazolium chloride; 1-[1,3-bis(hydroxymethyl)-2,5-dioxoimidazolidin-4-yl]-1,3-bis(hydroxymethyl)urea; N,N″-bis(hydroxymethyl)urea; reaction products with 2-(2-butoxyethoxy)ethanol, ethylene glycol and formaldehyde; 5-chloro-2-(4-chlorophenoxy)phenol; 2-butylbenzene[d]isothiazol-3-one; cis-1-(3-chloroallyl)-3,5,7-triaza-1-azoniaadamantane chloride; 3-benzo(b)thien-2-yl-5,6-dihydro-1,4,2-oxathiazine 4-oxide; reaction product of diisopropanolamine with formaldehyde (1:4); silver-zinc-aluminum-boron phosphate glass/glass oxide (silver- and zinc-containing); cyclohexylhydroxydiazene 1-oxide potassium salt and bis[1-cyclohexyl-1,2-di(hydroxy-κ-O)diazeniumato(2-)]-copper; 4,5-trimethylene-2-methylisothiazolin-3-one; zinc pyrithione; IPBC; triclosan; tetramethylolacetylenediurea; N,N′-dimethylolurea; N-methylolurea; dimethylol-dimethylhydantoin; N-methylolchloroacetamide; reaction products of allantoin; ethylene glycol formal; butyl diglycol formal and benzyl formal; captan; folpet; metam-sodium; nabam; thiabendazole; dazomet; chlorothalonil; carbendazim; propiconazole; TCMTB (2-benzothiazolylthio)methyl thiocyanate); α-cypermethrin; cyfluthrin; permethrin; fipronil; imidacloprid; thiametoxam; metaflumizone; amitraz and abamectin (avermectin).

20. The rodenticidal mixture according to claim 13, wherein the biocide comprises a mixture of one or more bactericides, one or more fungicides and one or more insecticides.

21. A rodenticidal bait formulation comprising
(A) 0.001 to 30% by weight (based on the total of (A), (B) and (C)) of a rodenticidal mixture according to claim 13,
(B) 5 to 99.999% by weight (based on the total of (A), (B) and (C)) of at least one bait material and
(C) 0 to 94.999% by weight (based on the total of (A), (B) and (C)) of one or more adjuvants.

22. The rodenticidal bait formulation according to claim 21, wherein the bait material comprises a cereal bait (CB) in the form of grains, coarse meal or powder, pellets, wax-coated pellets, molten-wax blocks, compressed or extruded wax blocks, pastes, gels, granules or foams.

23. A method of controlling rodent pests, comprising applying a rodenticidal bait formulation according to claim 21 in the rodent pests' habitat.

24. A bait box, comprising a rodenticidal bait formulation according to claim 21.
